(19) United States
(12) Patent Application Publication
(10) Pub. No.: US 2025/0207853 A1
(43) Pub. Date: Jun. 26, 2025

(54) PROCESSES FOR RECOVERY OF ONE OR MORE OF C2, C3, OR C4 OLEFINS FROM A PRODUCT STREAM OF OLEFIN PRODUCTION REACTOR SYSTEMS

(71) Applicant: Dow Global Technologies LLC, Midland, MI (US)

(72) Inventors: Adam M. McNeeley, Christianburg, VA (US); Yue Zhang, Freeport, TX (US); Matthew T. Pretz, Freeport, TX (US); John H. Holderness, Freeport, TX (US); Bahareh Sayyer, Freeport, TX (US)

(73) Assignee: Dow Global Technologies LLC, Midland, MI (US)

(21) Appl. No.: 18/849,013

(22) PCT Filed: Mar. 21, 2023
(86) PCT No.: PCT/US2023/064752

§ 371 Date: Sep. 20, 2024

Related U.S. Application Data
(60) Provisional application No. 63/322,825, filed on Mar. 23, 2022.

(51) Int. Cl. F25J 3/02; C10G 70/04

(52) U.S. Cl. CPC F25J 3/0242; C10G 70/043; C10G 2300/1081; C10G 2300/4081; F25J 2200/02; F25J 2200/74; F25J 2245/02

(57) ABSTRACT

A process for recovery of olefins includes flashing at least a portion of an alkane feed stream to produce a flashed alkane feed stream, introducing the flashed alkane feed stream in a cold box, a fractionation system, or both as cooling medium, compressing and cooling a gaseous feed stream, separating a compressed and cooled feed stream into first residual vapor stream and first liquid residue stream, cooling the first residual vapor stream, separating a cooled first residual vapor stream into a second vapor residue stream and a second liquid residue stream, separating the first liquid residue stream into an overhead stream of the light removal column and a bottom stream of the light removal column, and fractionating at least a portion of the second liquid residue stream and the bottom stream of the light removal column to produce overhead vapor stream, liquid recycle stream, and bottom liquid stream.

### CROSS-REFERENCE TO RELATED APPLICATIONS

This application claims the benefit of U.S. Provisional Application Ser. No. 63/322,825 filed Mar. 23, 2022, the entire disclosure of which is hereby incorporated by reference.

### TECHNICAL FIELD

Embodiments described herein generally relate to chemical processing and, more particularly, to product recovery in chemical processing.

### BACKGROUND

Ethylene, ethane, propylene, propane, and/or heavier hydrocarbons can be recovered from a variety of gas streams including, natural gas, refinery gas, synthetic gas, or combinations thereof, obtained from coal, crude oil, naphtha, oil shale, steam cracker, catalytic cracker, or combinations thereof. The cryogenic expansion processes are widely used for recovery of condensable product gas from non-condensable or difficult to condense gas as it provides start-up simplicity, operating flexibility, good efficiency and reliability.

In conventional cryogenic expansion recovery processes, the pressurized feed gas stream is cooled by heat exchange with other streams of process and/or external sources of refrigeration, such as ethylene or propylene compression-refrigeration systems. As the gas stream is cooled, liquid streams may be collected in one or more separators as high pressure liquids containing some of the desired C₂₊ components. Depending on the product richness of the liquid, this liquid stream may be expanded to a lower pressure and fractionated. This expansion, coupled with work extraction, lowers the temperature of the stream. Under some conditions, precooling of the pressurized liquid prior to these expansions may be desirable to further lower the temperature resulting from the expansion. The expanded stream is then fractioned through a distillation column (s) where the expanded cool streams are distilled to separate residual methane, hydrogen, non-condensable from one or more of desired C₂ or C₃, or heavier products.

### SUMMARY

Despite conventional recovery processes available for recovering ethylene, ethane, propylene, propane, and/or heavier hydrocarbons from variety of gas streams, these processes often require external refrigeration systems, which may decrease the efficiency of recovery and increase the cost.

Accordingly, there is an ongoing need for processes providing improved energy efficient recovery of one or more of C₂, C₃, or heavier products with reduced capital investment. These needs are met by embodiments of systems and processes described in the present disclosure. Embodiments of the present disclosure are directed to processes for recovery of one or more of C₂, C₃, or C₄ olefins from a product stream of an olefin production reactor system. Disclosed herein is the use of the refrigeration duty of the feed stream to the upstream alkane to alkene conversion process. For example, liquefied natural gas may be used as the feed to the alkane to alkene conversion plant. This liquefied natural gas may be utilized to cool various process streams in the downstream recovery unit. The processes of the present disclosure may utilize this refrigeration duty by including a light removal column and a fractionation system downstream of the light removal column, which produce streams that may be used as cooling mediums in the recovery processes. Additionally, the refrigeration duty may be incorporated into a cold box of the recovery system. With one or more of the cold box integration, the light removal column and the two-stage fractionation system, the processes of the present disclosure may not require as large of an external refrigeration system and recover one or more of C₂, C₃, or C₄ olefins with improved efficiency while reducing capital investment.

According to one or more embodiments disclosed herein, a process for recovery of one or more of C₂, C₃, or C₄ olefins from a product stream of an olefin production reactor system includes flashing at least a portion of an alkane feed stream of the olefin production reactor system to produce a flashed alkane feed stream, introducing the flashed alkane feed stream in a cold box, a fractionation system, or both as cooling medium, and compressing and cooling a gaseous feed stream to produce a compressed and cooled feed stream. The gaseous feed stream comprises at least 70 weight percent (wt. %) of the combination of C₂, C₃, and C₄ components. The process further includes separating the compressed and cooled feed stream into a first residual vapor stream and a first liquid residue stream, cooling the first residual vapor stream in the cold box to produce a cooled first residual stream, separating the cooled first residual stream into a second vapor residue stream and a second liquid residue stream, and separating the first liquid residue stream in a light removal column into an overhead stream of the light removal column and a bottom stream of the light removal column. The removal of light components may enable using the flashed alkane feed stream in the fractionation system as cooling medium. The process further includes fractionating at least a portion of the second liquid residue stream and the bottom stream of the light removal column in the fractionation system, to produce an overhead vapor stream, a liquid recycle stream, and a bottom liquid stream.

It is to be understood that both the foregoing brief summary and the following detailed description present embodiments of the technology, and are intended to provide an overview or framework for understanding the nature and character of the technology as it is claimed. The accompanying drawings are included to provide a further understanding of the technology, and are incorporated into and constitute a part of this specification. The drawings illustrate various embodiments and, together with the description, serve to explain the principles and operations of the technology. Additionally, the drawings and descriptions are meant to be merely illustrative, and are not intended to limit the scope of the claims in any manner.

Additional features and advantages of the technology disclosed herein will be set forth in the detailed description that follows, and in part will be readily apparent to those skilled in the art from that description or recognized by practicing the technology as described herein, including the detailed description that follows, the claims, as well as the appended drawings.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 schematically depicts a system for recovery of one or more of C₂, C₃, or C₄ olefins from a product stream of an olefin production reactor system, according to one or more embodiments described herein; and

FIG. 2 schematically depicts a two-stage fractionation system for recovery of one or more of C₂, C₃, or C₄ olefins from a product stream of an olefin production reactor system, according to one or more embodiments described herein.

It should be understood that the drawings are schematic in nature, and do not include some components of a fluidized catalyst processing system commonly employed in the art, such as, without limitation, temperature transmitters, pressure transmitters, flow meters, pumps, valves, and the like. It would be known that these components are within the spirit and scope of the present embodiments disclosed. However, operational components, such as those described in the present disclosure, may be added to the embodiments described in this disclosure.

Reference will now be made in greater detail to various embodiments, some embodiments of which are illustrated in the accompanying drawings. Whenever possible, the same reference numerals will be used throughout the drawings to refer to the same or similar parts.

### DETAILED DESCRIPTION

As used in this disclosure, a “separator” refers to any separation device or system of separation devices that at least partially separates one or more chemicals that are mixed in a process stream from one another. For example, a separator may selectively separate differing chemical species, phases, or sized material from one another, forming one or more chemical fractions. Examples of separators include, without limitation, distillation columns, flash drums, knock-out drums, knock-out pots, centrifuges, cyclones, filtration devices, traps, scrubbers, expansion devices, membranes, solvent extraction devices, and the like. It should be understood that separation processes described in this disclosure may not completely separate all of one chemical constituent from all of another chemical constituent. It should be understood that the separation processes described in this disclosure “at least partially” separate different chemical components from one another, and that even if not explicitly stated, it should be understood that separation may include only partial separation. As used in this disclosure, one or more chemical constituents may be “separated” from a process stream to form a new process stream. Generally, a process stream may enter a separator and be divided, or separated, into two or more process streams of desired composition.

As used in this disclosure, a “cold box” refers to one or more heat exchangers connected in series. The heat exchangers may include a brazed heat exchanger, a shell and tube heat exchanger, double pipe heat exchanger, plate heat exchanger, tubular heat exchanger, fin type heat exchanger, condensers, evaporators, boilers, or combinations thereof.

As used in this disclosure, a “fractionation system” refers to any fractionation device or system of fractionation devices that at least partially divide a certain quantity of a mixture (gas, solid, liquid, or combinations thereof), during a phase transition, into a number of smaller fractions in which the composition varies according to a gradient.

It should further be understood that streams may be named for the components of the stream, and the component for which the stream is named may be the major component of the stream (such as comprising from 50 weight percent (wt. %), from 70 wt. %, from 90 wt. %, from 95 wt. %, from 99 wt. %, from 99.5 wt. %, or even from 99.9 wt. % of the contents of the stream to 100 wt. % of the contents of the stream). It should also be understood that components of a stream are disclosed as passing from one system component to another when a stream comprising that component is disclosed as passing from that system component to another.

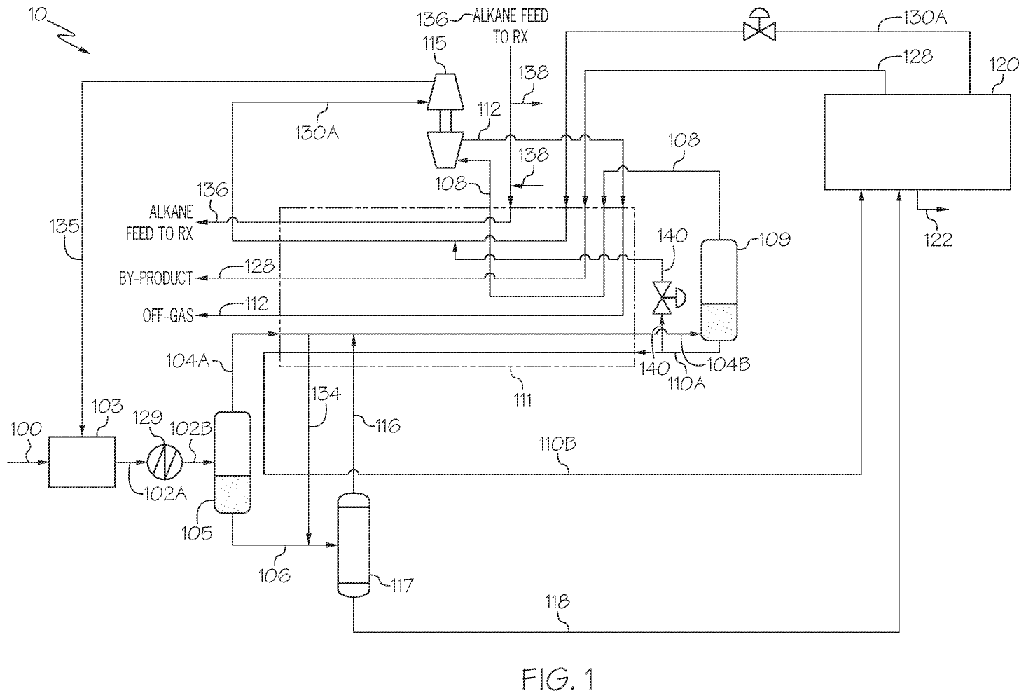

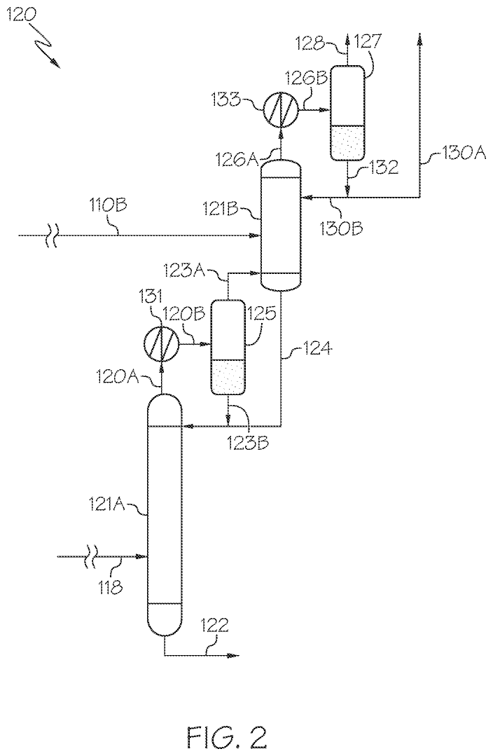

Embodiments of the present disclosure are directed to processes for recovering one or more of C₂, C₃, or C₄ olefins from a product stream of an olefin production reactor system. Such processes utilize systems that have particular features, such as a particular orientation of system parts. FIGS. 1 and 2 depicts such a system 10, which includes a cold box 111, a light removal column 117, and a fractionation system 120.

Referring now to FIG. 1, a system 10 for recovering one or more of C₂, C₃, or C₄ olefins from a product stream of an olefin production reactor system is schematically depicted. The system 10 generally receives a gaseous feed stream 100 and directly processes the gaseous feed stream 100 to recover one or more of C₂, C₃, or C₄ olefins from a product stream of an olefin production reactor system.

The gaseous feed stream 100 may be introduced to the system 10 for recovering one or more of C₂, C₃, or C₄ olefins from a product stream of an olefin production reactor system. The product stream may be from a petrochemical process or naphtha from a refining operation for crude oil, natural gas liquids (NGL), or other hydrocarbon sources. The product stream may include the gaseous feed stream 100. As described herein, in one or more embodiments, the gaseous feed stream 100 may be a reaction effluent of steam cracking, catalytic cracking, or both. In one or more embodiments, the gaseous feed stream 100 may include natural gas, refinery gas, synthetic gas, or combinations thereof obtained from coal, crude oil, naphtha, oil shale, or combinations thereof. In embodiments, the gaseous feed stream 100 may include at least 70 wt. %, at least 75 wt. %, at least 80 wt. %, or at least 85 wt. % of one or more of C₂, C₃, or C₄ components. In embodiments, the gaseous feed stream 100 comprises from 1 wt. % to 10 wt. % N₂, from 0.01 wt. % to 5 wt % H₂, from 0.01 wt. % to 5 wt. % methane, and from 70 wt. % to 95 wt. % one or more of C₂, C₃, or C₄ components. In embodiments, the gaseous feed stream 100 comprises ethane, propane, butane, or combinations thereof. In embodiments, the gaseous feed stream 100 comprises 50 wt. % propane.

The gaseous feed stream 100 may be introduced to the compressor 103. The gaseous feed stream 100 may be compressed in the compressor 103 to produce the compressed feed stream 102A. The compressed feed stream 102A may be introduced to the heat exchanger 129. The amount of one or more of C₂, C₃, or C₄ components in the compressed feed stream 102A may be greater than the amount of one or more of C₂, C₃, or C₄ components in the gaseous feed stream 100. The compressed feed stream 102A may be cooled in the heat exchanger 129 to produce the compressed and cooled feed stream 102B. In embodiments, the compressed feed stream 102A may be cooled in the heat exchanger 129 by cooling water, a bottom liquid stream 122, or both. When the compressed feed stream 102A is cooled by the bottom liquid stream 122, the cooling required in the heat exchanger 129 may be provided by heating, flashing, vaporizing the bottom liquid stream 122, or combinations thereof. In embodiment, the bottom liquid stream 122 may be flashed before entering to the heat exchanger 129. The bottom liquid stream 122 may be flashed to enable favorable cooling temperature ranges. When the cooling is provided by vaporizing the bottom liquid stream 122, from 10 wt. % to 99.9 wt. %, from 10 wt. % to 99 wt. %, from 10 wt. % to 95 wt. %, from 10 wt. % to 90 wt. %, from 10 wt. % to 85 wt. %, from 10 wt. % to 80 wt. %, from 15 wt. % to 99.9 wt. %, from 15 wt. % to 99 wt. %, from 15 wt. % to 95 wt. %, from 15 wt. % to 90 wt. %, from 15 wt. % to 85 wt. %, from 15 wt. % to 80 wt. %, from 20 wt. % to 99.9 wt. %, from 20 wt. % to 99 wt. %, from 20 wt. % to 95 wt. %, from 20 wt. % to 90 wt. %, from 20 wt. % to 85 wt. %, from 20 wt. % to 80 wt. %, from 25 wt. % to 99.9 wt. %, from 25 wt. % to 99 wt. %, from 25 wt. % to 95 wt. %, from 25 wt. % to 90 wt. %, from 25 wt. % to 85 wt. %, or from 25 wt. % to 80 wt. % of the bottom liquid stream 122 may be vaporized.

The compressed and cooled feed stream 102B may be introduced to a first separator 105. The compressed and cooled feed stream 102B may be separated into a first residual vapor stream 104A and a first liquid residue stream 106. In embodiments, the first residual vapor stream 104A may comprise from 1 wt. % to 25 wt. % N₂, from 0.01 wt. % to 5 wt. % H₂, from 0.01 wt. % to 5 wt. % methane, and from 70 wt. % to 95 wt. % one or more of C₂, C₃, or C₄ components. The amount of one or more of C₂, C₃, or C₄ components in the first residual vapor stream 104A may be greater than the amount of one or more of C₂, C₃, or C₄ components in the compressed feed stream 102A.

The first residual vapor stream 104A may be introduced to a cold box 111. The first residual vapor stream 104A may be cooled in the cold box 111 to produce the cooled first residual vapor stream 104B. In embodiments, the cooled first residual vapor stream 104B may comprise from 1 wt. % to 45 wt. % N₂, from 0.01 wt. % to 15 wt. % H₂, from 0.01 wt. % to 5 wt. % methane, and from 50 wt. % to 95 wt. % one or more of C₂, C₃, or C₄ components.

The cooled first residual vapor stream 104B may be introduced to a second separator 109. The cooled first residual vapor stream 104B may be separated into a second vapor residue stream 108 and a second liquid residue stream 110A. In embodiments, the second vapor residue stream 108 comprises from 30 wt. % to 80 wt. % N₂, from 10 wt. % to 40 wt. % H₂, from 5 wt. % to 20 wt. % methane, and from 0.01 wt. % to 10 wt. % one or more of C₂, C₃, or C₄ components. In embodiments, the second vapor residue stream 108 may have a temperature of from −120° C. to −100° C. In embodiments, the second liquid residue stream 110A may comprise from 0.001 wt. % to 2 wt. % N₂, from 0.001 wt. % to 1 wt. % H₂, from 0.001 wt. % to 2 wt. % methane, and from 90 wt. % to 99 wt. % one or more of C₂, C₃, or C₄ components.

The second vapor residue stream 108 may be introduced to the cold box 111. At least a portion of the second vapor residue stream 108 may be passed through the cold box 111 and then introduced to the turbo-expander/compressor 115. The at least a portion of the second vapor residue stream 108 may be expanded at the turbo-expander/compressor 115 to produce an expanded second vapor residue stream 112. In embodiments, the expanded second vapor residue stream 112 may comprise from 40 wt. % to 90 wt. % N₂, from 10 wt. % to 50 wt. % H₂, from 1 wt. % to 10 wt. % methane, and from 1 wt. % to 10 wt. % one or more of C₂, C₃, or C₄ components. In embodiments, the expanded second vapor residue stream 112 may have a temperature of from 0° C. to 50° C. The expanded second vapor residue stream 112 may be introduced to the cold box 111. The expanded second vapor residue stream 112 may be used as a cooling medium in the cold box 111. The expanded second vapor residue stream 112 may be passed through the cold box 111 and exit the system 10 as off-gas.

Still referring to FIG. 1, the second liquid residue stream 110A may be introduced to the cold box 111 downstream of the first separator 105. The second liquid residue stream 110A may be used as a cooling medium in the cold box 111. The second liquid residue stream 110A may be cooled at the cold box 111 to produce a cooled second liquid residue stream 110B. In embodiments, the cooled second liquid residue stream 110B may comprise from 0.001 wt. % to 5 wt. % N₂, from 0.001 wt. % to 1 wt. % H₂, from 0.001 wt. % to 1 wt. % methane, and from 90 wt. % to 99 wt. % one or more of C₂, C₃, or C₄ components.

At least a portion of the second liquid residue stream 140 may be introduced to the cold box 111. The at least a portion of the second liquid residue stream 140 may comprise from 0.001 wt. % to 2 wt. % N₂, from 0.001 wt. % to 1 wt. % H₂, from 0.001 wt. % to 2 wt. % methane, and from 90 wt. % to 99 wt. % one or more of C₂, C₃, or C₄ components. The at least a portion of the second liquid residue stream 140 may be mixed with a liquid recycle stream 130A in the cold box 111. The at least a portion of the second liquid residue stream 140 may be used as a cooling medium in the cold box 111.

Still referring to FIG. 1, the first liquid residue stream 106 may be introduced to the light removal column 117 downstream of the first separator 105. The light removal column 117 may be disposed upstream of the two-stage fractionation system. The light removal column 117 may enable removal of light and non-condensable components comprising N₂, H₂, methane, or combinations thereof, from the first liquid residue stream 106 upstream of the fractionation system 120 to produce an overhead stream of the light removal column 116 and a bottom stream of the light removal column 118. The second overhead stream 126A may be separated to produce various streams that may be used as cooling mediums in the cold box 111. In the light removal column 117, the first liquid residue stream 106 may be separated into the overhead stream of the light removal column 116 and the bottom stream of the light removal column 118. In embodiments, the overhead stream of the light removal column 116 may comprise from 1 wt. % to 30 wt. % N₂, from 1 wt. % to 10 wt. % H₂, from 0.01 wt. % to 5 wt. % methane, and from 70 wt. % to 95 wt. % one or more of C₂, C₃, or C₄ components. The overhead stream of the light removal column 116 may be mixed with the first residual vapor stream 104A at the cold box 111 and then introduced to the second separator 109. In embodiments, the bottom stream of the light removal column 118 may comprise at least 95 wt. %, at least 96 wt. %, at least 97 wt. %, at least 98 wt. %, at least 99 wt. %, or at least 99.9 wt. % one or more of C₂, C₃, or C₄ components. The bottom stream of the light removal column 118 may include less than 1 wt. %, less than 0.1 wt. %, less than 0.01 wt. %, less than 0.001 wt. %, or even less than 0.0001 wt. % N₂, H₂, methane, or combinations thereof. The bottom stream of the light removal column 118 may not include H₂. The bottom stream of the light removal column 118 may be introduced to the fractionation system 120.

Advantageously, the removal of light and non-condensable components by the light removal column 117 may enable an increased and more favorable temperature of the overhead stream 126A in the fractionation system 120 since the lightest components of first liquid residue stream 106 are removed and not passed to the fractionation system. This higher temperature of overhead stream 126A may be cooled for condensation in the heat exchanger 133 without the need for external refrigeration systems. Instead, the flashed alkane feed stream 138 may be adequate to sufficiently cool overhead stream 126A whereas without the use of the light removal column 117, external refrigeration systems may be necessary.

At least the portion of the second liquid residue stream 110A, at least the portion of the bottom stream of the light removal column 118, or both, may be introduced to the fractionation system 120 to produce an overhead vapor stream 128, a liquid recycle stream 130A, and a bottom liquid stream 122. Referring to FIGS. 1 and 2, one example of the fractionation system 120 is a two-stage fractionation system, which includes the first fractionator 121A and the second fractionator 121B downstream of the first fractionator 121A. The two-stage fractionation system may enable utilization of cooling water for compression duty of a first overhead stream 120A from the first fractionator 121A. As a result of removal of light and non-condensable components by the light removal column 117, the gaseous feed stream 100 may be compressed using cooling water and heat integration from the first overhead stream 120A. Utilization of cooling water may be beneficial to the utilization of a heat rejection refrigerant process due to superior thermodynamic efficiency as well as low associated capital cost. Additionally, the two-stage fractionation system may produce various streams that may be used as cooling mediums in the cold box. The two-stage fractionation system may provide flexibility in operation and product specification adjustment.

Still referring to FIGS. 1 and 2, in embodiments, at least the portion of the second liquid residue stream 110A may be introduced to the first fractionator 121A depending on stage compositions. In embodiments, at least the portion of the second liquid residue stream 110A may be cooled in the cold box 111 to produce the cooled second liquid residue stream 110B. The cooled second liquid residue stream 110B may be introduced to the fractionation system 120. In embodiments, the cooled second liquid residue stream 110B may be introduced to the first fractionator 121A of the two-stage fractionation system. As discussed above, the bottom stream of the light removal column 118 may be introduced to the first fractionator 121A. At least a portion of the cooled second liquid residue stream 110B and the bottom stream of the light removal column 118 may be separated into a first overhead stream 120A and a bottom liquid stream 122. In embodiments, when the cooled second liquid residue stream 110B is fed to the first fractionator 121A, the cooled second liquid residue stream 110B and the bottom stream of the light removal column 118 may be separated into a first overhead stream 120A and a bottom liquid stream 122.

In embodiments, the first overhead stream 120A comprises from 0.001 to 1 wt. % N₂, from 0 to 1 wt. % H₂, from 0.001 to 1 wt. % methane, and from 98 wt. % to 99.9 wt. % one or more of C₂, C₃, or C₄ components. The first overhead stream 120A may not include H₂. In embodiments, the bottom liquid stream 122 may comprise at least 95 wt. %, at least 96 wt. %, at least 97 wt. %, at least 98 wt. %, at least 99 wt. %, or at least 99.9 wt. % one or more of C₂, C₃, or C₄ components. The bottom liquid stream 122 may not include N₂, H₂, methane, or combinations thereof.

The first overhead stream 120A may be partially condensed and refluxed. The temperature of the first overhead stream 120A may be controlled to enable utilizing cooling water as a condensing agent for the gaseous feed 100. In embodiments, the first overhead stream 120A may be cooled by the heat exchanger 131 to produce a cooled first overhead stream 120B. In embodiments, the heat exchanger 131 may use cooling water as a cooling medium. The heat exchanger 131 may not require external refrigeration system. The cooled first overhead stream 120B may be introduced to the third separator 125. The cooled first overhead stream 120B may be separated into a first vapor stream 123A and a first liquid stream 123B. The first liquid stream 123B may be reintroduced into the first fractionator 121A.

Still referring to FIGS. 1 and 2, at least a portion of the first overhead stream 120A may be introduced to the second fractionator 121B downstream of the first fractionator 121A and then separated into a second overhead stream 126A and a second bottom stream 124. In embodiments, the first vapor stream 123A including non-condensable components may be introduced to the second fractionator 121B. The first vapor stream 123A may be separated into the second overhead stream 126A and the second bottom stream 124. In embodiments, the second bottom stream 124 may comprise at least 95 wt. %, at least 96 wt. %, at least 97 wt. %, at least 98 wt. %, or at least 99 wt. % one or more of C₂, C₃, or C₄ components. The second bottom stream 124 may further comprise from 0 wt. % to 1 wt. % H₂, from 0 wt. % to 1 wt. % N₂, and from 0.001 wt. % to 1 wt. % methane. The second bottom stream 124 may not include H₂, N₂, or both. The second bottom stream 124 may be reintroduced into the first fractionator 121A.

The second overhead stream 126A may be partially condensed and refluxed. The temperature of the second overhead stream 126A may be controlled using the alkane feed stream 136 to meet the desired specification of total product loss. In embodiments, the second overhead stream 126A may be cooled by the heat exchanger 133 to produce a cooled second overhead stream 126B. In embodiments, the second overhead stream 126A may be cooled in the heat exchanger 133 with the flashed alkane feed stream 138. In embodiments, the heat exchanger 133 may be used a branch of the flashed alkane feed stream 138, as a refrigerant (cooling medium), and thereby may not require external refrigeration system.

The cooled second overhead stream 126B may be introduced to a fourth separator 127, and then separated into an overhead vapor stream 128 and an overhead liquid stream 132. In embodiments, the overhead vapor stream 128 may comprise from 1 wt. % to 10 wt. % N₂, from 0.001 wt. % to 1 wt. % H₂, from 1 wt. % to 10 wt. % methane, and from 75 wt. % to 95 wt. % one or more of C₂, C₃, or C₄ components.

The overhead vapor stream 128 may be introduced to the cold box 111. The overhead vapor stream 128 may be cooled in the cold box 111 to produce by-products. In embodiments, the overhead vapor stream 128 may be used as a cooling medium in the cold box 111 before existing the system 10 as by-products.

The overhead liquid stream 132 may be split into a liquid recycle stream 130A and a liquid reflux return stream 130B. The liquid recycle stream 130A may be introduced to the cold box 111. The liquid recycle stream 130A may be used as a cooling medium in the cold box. The liquid reflux return stream 130B may be introduced to the second fractionator 121B.

In embodiments, the liquid recycle stream 130A may comprise from 0.001 wt. % to 1 wt. % N₂, from 0 wt. % to 1 wt. % H₂, from 0.001 wt. % to 1 wt. % methane, and from 90 wt. % to 99 wt. % one or more of C₂, C₃, or C₄ components. In embodiments, the liquid recycle stream 130A may not include H₂.

Still referring to FIGS. 1 and 2, at least a portion of the liquid recycle stream 130A may be introduced to the turbo-expander/compressor 115. Heat may be recovered from the liquid recycle stream 130A, and the at least a portion of the liquid recycle stream 130A may be compressed at the turbo-expander/compressor 115 to produce a recycling stream 135. The recycling stream 135 may be introduced to the compressor 103 to compress with the gaseous feed stream 100.

The second bottom stream 124 may be re-introduced to the first fractionator 121A. Prior to re-introducing to the first fractionator 121A, the second bottom stream 126 may be mixed with the first liquid stream 123B.

Still referring to FIGS. 1 and 2, in embodiments, the alkane feed stream 136 may be introduced to the system 10 as a medium of heat exchange. As described herein, an “alkane feed stream” may include alkanes, such as in an amount greater than 25 wt. %, 50 wt. %, 75 wt. %, or even 95 wt. %. In embodiments, the alkane feed stream 136 may be converted to the gaseous feed stream 100 by a reactor system not shown. For example, the reactor system may dehydrogenate the alkane feed stream 136 to form the gaseous feed stream 100.

The alkane feed stream 136 may be a liquid stream which is relatively cold. In embodiments, at least a portion of the alkane feed stream 106 may be flashed to produce a flashed alkane feed stream 138. In embodiments, at least a portion of the alkane feed stream 136 may be flashed at a pressure of from −5 pound-force per square inch (psig) (0.07 megapascal(MPa)) to 136 psig (1.04 MPa), from −5 psig (0.07 MPa) to 106 psig (0.83 MPa), from 5 psig (0.14 MPa) to 106 psig (0.83 MPa), 5 psig (0.14 MPa) to 136 psig (1.04 MPa), 25 psig (0.27 MPa) to 136 psig (1.04 MPa), 25 psig (0.27 MPa) to 106 psig (0.83 MPa), or from 25 psig (0.27 MPa) to 46 psig (0.42 MPa) and vaporized at a temperature of from −25° C. to −5° C., −25° C. to 0° C., −25° C. to 40° C., −20° C. to −5° C., −20° C. to 0° C., −20° C. to 40° C., −15° C. to −5° C., −15° C. to 0° C., or −15° C. to 40° C. prior routing to a reaction area. For a vapor phase reaction at relatively low pressure, the alkane feed stream 136 may be flashed to a relatively low pressure and vaporized at refrigeration temperatures prior routing to a reaction area.

The alkane feed stream 136 may be introduced to the cold box 111, the fractionation system 120, or both and used as a cooling medium in the cold box 111, the fractionation system 120, or both. In embodiments, the flashed alkane feed stream 138 may be introduced to the cold box 111, the fractionation system 120, or both and used as a cooling medium. The available refrigeration duty from the alkane feed stream 136 at these refrigeration temperatures may drive several sections of the cold box 111 and eliminate the requirement for external refrigeration systems. In addition, the flashed alkane feed stream 138 may be used as refrigerant in the heat exchanger 133 and eliminate the requirement for external refrigeration systems. In such embodiments, the refrigerant duty of the alkane feed stream 136 is essentially “free” to the system since the alkane feed stream 136 must be heated to some degree prior to it being converted to the gaseous feed stream 100.

A process for recovery of one or more of C₂, C₃, C₄ olefins, or combinations of these, from a product stream of an olefin production reactor system of the present disclosure may produce greater yield of one or more of C₂, C₃, or C₄ olefins, compared to a conventional recovery process. In embodiments, the process of the present disclosure may produce a combined yield of C₂, C₃, C₄ olefins, or combinations of these, of greater than or equal to 90 wt. %, greater than or equal to 95 wt. %, greater than or equal to 96 wt. %, greater than or equal to 97 wt. %, greater than or equal to 98 wt. %, or even greater than or equal to 99 wt. %, based on the total weight of the product stream introduced to the recovery process of the present disclosure.

### EXAMPLES

The various embodiments of processes and systems for recovery of the conversion of one or more of C₂, C₃, or C₄ olefins from a product stream of an olefin production reactor system will be further clarified by the following examples. The examples are illustrative in nature, and should not be understood to limit the subject matter of the present disclosure.

### Example 1

Example 1 was conducted using an integrated separation train process model in Aspen Plus for the recovery systems depicted in FIGS. 1 and 2 where the propane feedstock at Gulf coast was provided. Table 1 shows a temperature, a pressure, and a mass fraction ratio of streams depicted in FIGS. 1 and 2.

TABLE 1

Stream in FIGS. 1 and 2

| | 100 | 102 | 104Λ | 104B | 108 | 110Λ | 110B | 112 |
|---|---|---|---|---|---|---|---|---|
| Temperature (° C.) | 44 | 114 | 27 | −103 | −103 | −103 | 21 | 21 |
| Pressure (psig) | 6 | 420 | 415 | 407 | 407 | 407 | 401 | 156 |
| N₂ | 2.0% | 2.0% | 4.5% | 14.8% | 45.2% | 0.6% | 0.3% | 45.2% |
| H₂ | 1.7% | 1.6% | 3.8% | 12.6% | 39.3% | 0.1% | 0.1% | 39.3% |
| CH₄ | 0.4% | 0.4% | 0.9% | 2.8% | 7.1% | 0.7% | 0.3% | 7.1% |
| C₂ | 3.7% | 6.6% | 9.9% | 18.1% | 5.1% | 24.1% | 12.2% | 5.1% |
| C₃ | 87.3% | 87.9% | 79.6% | 48.7% | 1.0% | 71.1% | 85.7% | 1.0% |
| C₄₊ | 0.4% | 0.4% | 0.1% | 0.0% | 0.0% | 0.0% | 0.0% | 0.0% |

Stream in FIGS. 1 and 2

| | 106 | 116 | 118 | 120A | 122 | 124 | 128 |
|---|---|---|---|---|---|---|---|
| Temperature (° C.) | 27 | 22 | 62 | 49 | 70 | 39 | 21 |
| Pressure (psig) | 415 | 412 | 413 | 387 | 394 | 383 | 148 |
| N₂ | 0.2% | 6.6% | 0.1% | 0.1% | 0.0% | 0.0% | 2.6% |
| H₂ | 0.1% | 4.0% | 0.0% | 0.0% | 0.0% | 0.0% | 0.4% |
| CH₄ | 0.1% | 1.1% | 0.1% | 0.2% | 0.0% | 0.1% | 2.5% |
| C₂ | 4.2% | 10.8% | 4.5% | 27.8% | 0.0% | 23.3% | 85.3% |
| C₃ | 94.6% | 76.0% | 94.6% | 71.1% | 99.5% | 75.9% | 2.0% |
| C₄₊ | 0.6% | 0.0% | 0.5% | 0.0% | 0.4% | 0.0% | 0.0% |

| | Stream in FIGS. 1 and 2 | | |
|---|---|---|---|
| | 130A | 140 | 136 |
| Temperature (° C.) | −5 | −103 | 21 |
| Pressure (psig) | 380 | 407 | 27 |
| N₂ | 0.3% | 0.6% | 0.0% |
| H₂ | 0.0% | 0.1% | 0.0% |
| CH₄ | 0.6% | 0.7% | 0.0% |
| C₂ | 90.0% | 24.1% | 2.1% |
| C₃ | 5.5% | 71.1% | 97.8% |
| C₄₊ | 0.0% | 0.0% | 0.1% |

As shown in Example 1, one or more of C₂, C₃, or C₄ was energy efficient recovered by utilizing cold box, the light removal column, and the two-stage fractionation system. Example 1 further used liquefied natural gas as the feed to the alkane to alkene conversion plant and utilized the liquefied natural gas to cool various process streams in the downstream recovery unit.

According to a first aspect of the present disclosure, a process for recovery of one or more of C₂, C₃, or C₄ olefins from a product stream of an olefin production reactor system includes flashing at least a portion of an alkane feed stream of the olefin production reactor system to produce a flashed alkane feed stream, introducing the flashed alkane feed stream in a cold box, a fractionation system, or both as cooling medium, and compressing and cooling a gaseous feed stream to produce a compressed and cooled feed stream. The gaseous feed stream comprises at least 70 weight percent (wt. %) of the combination of C₂, C₃, and C₄ components. The process further includes separating the compressed and cooled feed stream into a first residual vapor stream and a first liquid residue stream, cooling the first residual vapor stream in the cold box to produce a cooled first residual stream, separating the cooled first residual stream into a second vapor residue stream and a second liquid residue stream, and separating the first liquid residue stream in a light removal column into an overhead stream of the light removal column and a bottom stream of the light removal column. The removal of light components may enable using the flashed alkane feed stream in the fractionation system as cooling medium. The process further includes fractionating at least a portion of the second liquid residue stream and the bottom stream of the light removal column in the fractionation system, to produce an overhead vapor stream, a liquid recycle stream, and a bottom liquid stream.

A second aspect of the present disclosure may include the first aspect, the fractionation system may comprise a two-stage fractionation system. The process may further include fractionating at least a portion of the second liquid residue stream and the bottom stream of the light removal column in a first fractionator into a first overhead stream and the bottom liquid stream, and fractionating at least a portion of the first overhead stream in a second fractionator downstream of the first fractionator into a second overhead stream and a second bottom stream.

A third aspect of the present disclosure may include either the first or second aspects, further comprising cooling the first overhead stream to produce a cooled first overhead stream, separating the cooled first overhead stream into a first vapor stream and a first liquid stream, introducing the first vapor stream to the second fractionator, and reintroducing the first liquid stream and the second bottom stream to the first fractionator.

A fourth aspect of the present disclosure may include any of the first through third aspects, further comprising cooling the second overhead stream in a heat exchanger, separating the cooled second overhead stream into an overhead vapor stream and an overhead liquid stream, and splitting the overhead liquid stream into a liquid recycle stream and a liquid reflux return stream.

A fifth aspect of the present disclosure may include any of the first through fourth aspects, further comprising cooling the second overhead stream in a heat exchanger by heat exchange with the flashed alkane feed stream.

A sixth aspect of the present disclosure may include any of the first through fifth aspects, further comprising cooling at least a portion of the overhead vapor stream in the cold box to produce by-products.

A seventh aspect of the present disclosure may include any of the first through sixth aspects, further comprising introducing the liquid reflux return stream to the second fractionator, introducing the liquid recycle stream in the cold box, wherein the liquid recycle stream is used as a cooling medium in the cold box, compressing at least a portion of the liquid recycle stream to produce a recycle stream, and introducing the recycle stream to a compressor to compress with the gaseous feed stream.

An eighth aspect of the present disclosure may include any of the first through seventh aspects, further comprising removing light components comprising N₂, H₂, methane, or combinations thereof, from the first liquid residue stream in a light removal column to produce the overhead stream of the light removal column and the bottom stream of the light removal column.

A ninth aspect of the present disclosure may include any of the first through eighth aspects, further comprising cooling at least a portion of the second liquid residue stream in the cold box to produce a cooled second liquid residue stream, and introducing the cooled second liquid residue stream to the fractionation system.

A tenth aspect of the present disclosure may include any of the first through ninth aspects, further comprising introducing at least a portion of the second liquid residue stream to the cold box, wherein the second liquid residue stream is used as a cooling medium in the cold box.

An eleventh aspect of the present disclosure may include any of the first through tenth aspects, further comprising introducing the second vapor residue stream to the cold box, wherein the second vapor residue stream is used as a cooling medium in the cold box.

A twelfth aspect of the present disclosure may include any of the first through eleventh aspects, further comprising expanding at least a portion of the second vapor residue stream to produce an expanded second vapor residue stream, and introducing the expanded second vapor residue stream to the cold box, wherein the expanded second vapor residue stream is used as a cooling medium in the cold box

The subject matter of the present disclosure has been described in detail and by reference to specific embodiments. It should be understood that any detailed description of a component or feature of an embodiment does not necessarily imply that the component or feature is essential to the particular embodiment or to any other embodiment. Further, it should be apparent to those skilled in the art that various modifications and variations can be made to the described embodiments without departing from the spirit and scope of the claimed subject matter.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.” It should be understood that where a first component is described as “comprising” a second component, it is contemplated that, in some embodiments, the first component “consists” or “consists essentially of” that second component.

It should be understood that any two quantitative values assigned to a property may constitute a range of that property, and all combinations of ranges formed from all stated quantitative values of a given property are contemplated in this disclosure.

1. A process for recovery of one or more of C₂, C₃, or C₄ olefins from a product stream of an olefin production reactor system, the process comprising:
flashing at least a portion of an alkane feed stream of the olefin production reactor system to produce a flashed alkane feed stream;
introducing the flashed alkane feed stream in a cold box, a fractionation system, or both as cooling medium;
compressing and cooling a gaseous feed stream to produce a compressed and cooled feed stream, wherein the gaseous feed stream comprises at least 70 weight percent (wt. %) of the combination of C₂, C₃, and C₄ components;
separating the compressed and cooled feed stream into a first residual vapor stream and a first liquid residue stream;
cooling the first residual vapor stream in the cold box to produce a cooled first residual stream;
separating the cooled first residual stream into a second vapor residue stream and a second liquid residue stream;
separating the first liquid residue stream in a light removal column into an overhead stream of the light removal column and a bottom stream of the light removal column, where the removal of light components enables using the flashed alkane feed stream in the fractionation system as cooling medium; and
fractionating at least a portion of the second liquid residue stream and the bottom stream of the light removal column in the fractionation system, to produce an overhead vapor stream, a liquid recycle stream, and a bottom liquid stream.

2. The process of claim 1, where the fractionation system comprises a two-stage fractionation system, the process further comprises:
fractionating at least a portion of the second liquid residue stream, fractionating at least a portion of the bottom stream of the light removal column, or both, in a first fractionator into a first overhead stream and the bottom liquid stream; and
fractionating at least a portion of the first overhead stream in a second fractionator downstream of the first fractionator into a second overhead stream and a second bottom stream.

3. The process of claim 1, further comprising:
cooling the first overhead stream to produce a cooled first overhead stream;
separating the cooled first overhead stream into a first vapor stream and a first liquid stream;
introducing the first vapor stream to the second fractionator; and
reintroducing the first liquid stream and the second bottom stream to the first fractionator.

4. The process of claim 1, further comprising:
cooling the second overhead stream in a heat exchanger;
separating the cooled second overhead stream into an overhead vapor stream and an overhead liquid stream; and
splitting the overhead liquid stream into the liquid recycle stream and a liquid reflux return stream.

5. The process of claim 1, further comprising cooling the second overhead stream in a heat exchanger by heat exchange with the flashed alkane feed stream.

6. The process of claim 1, further comprising cooling at least a portion of the overhead vapor stream in the cold box to produce by-products.

7. The process of claim 1, further comprising:
introducing the liquid reflux return stream to the second fractionator;
introducing the liquid recycle stream in the cold box, wherein the liquid recycle stream is used as a cooling medium in the cold box;
compressing at least a portion of the liquid recycle stream to produce a recycle stream; and
introducing the recycle stream to a compressor to compress with the gaseous feed stream.

8. The process of claim 1, further comprising removing light components comprising N₂, H₂, methane, or combinations thereof, from the first liquid residue stream in a light removal column to produce the overhead stream of the light removal column and the bottom stream of the light removal column.

9. The process of claim 1, further comprising:
cooling at least a portion of the second liquid residue stream in the cold box to produce a cooled second liquid residue stream; and
introducing the cooled second liquid residue stream to the fractionation system.

10. The process of claim 1, further comprising introducing at least a portion of the second liquid residue stream to the cold box, wherein the second liquid residue stream is used as a cooling medium in the cold box.

11. The process of claim 1, further comprising introducing the second vapor residue stream to the cold box, wherein the second vapor residue stream is used as a cooling medium in the cold box.

12. The process of claim 1, further comprising:
expanding at least a portion of the second vapor residue stream to produce an expanded second vapor residue stream; and
introducing the expanded second vapor residue stream to the cold box, wherein the expanded second vapor residue stream is used as a cooling medium in the cold box.
